(19) United States
(12) Patent Application Publication
(10) Pub. No.: US 2016/0296127 A1
(43) Pub. Date: Oct. 13, 2016

(54) METHODS AND SYSTEM FOR ASSESSMENT OF PERIPHERAL PERFUSION

(71) Applicants: Jonathan Clark Roberts, Sammamish, WA (US); Damon Scott Pierce, Mercer Island, WA (US)

(72) Inventors: Jonathan Clark Roberts, Sammamish, WA (US); Damon Scott Pierce, Mercer Island, WA (US)

(21) Appl. No.: 15/096,535

(22) Filed: Apr. 12, 2016

Related U.S. Application Data
(60) Provisional application No. 62/146,869, filed on Apr. 13, 2015.

(51) Int. Cl. A61B 5/0295; A61B 5/00

(52) U.S. Cl. CPC A61B 5/0295; A61B 5/742; A61B 5/7465

(57) ABSTRACT

The present technology is directed to apparatuses, systems, and methods for assessing absolute and relative peripheral perfusion. In various embodiments, two blood volume measurement devices measure the blood volume at a patient's extremities either simultaneously or in series. Distortions between the waveforms generated by the two blood volume measurement devices are detected, and an assessment of the patient's perfusion is determined based on the degree of distortion.

### PRIORITY

This application claims priority to U.S. provisional application Ser. No. 62/146,869 filed on Apr. 13, 2015, the entirety of which is incorporated herein.

### TECHNICAL FIELD

The present technology relates generally to blood volume measurement and, more particularly, to techniques for utilizing blood volume measurements to assess peripheral perfusion. Additionally, the present technology relates to the delivery of these measurements to a physician for the purpose of remote monitoring the patient.

### BACKGROUND

Peripheral perfusion, or the adequacy of blood flow through the peripheral vasculature, can be assessed to detect deficits and potentially prevent limb or life threatening situations. Techniques for measuring for peripheral perfusion currently require a patient to visit a healthcare provider wherein the healthcare provider takes multiple blood volume or blood pressure measurements using a single measurement device. For example, the healthcare provider may measure blood pressure at a patient's arm. The healthcare provider may then measure blood pressure at the patient's ankle. The two blood pressures can then be analyzed to assess the patient's peripheral perfusion. These and other healthcare provider assessments, however, tend to have significant limitations including requiring costly and time-consuming in-facility testing by trained medical personnel using expensive equipment, infrequent testing for at-risk patients, and accuracy and repeatability issues. For example, the blood pressure measurements require the sequential use of blood pressure cuffs by a skilled technician in order to ensure accuracy of the measurements.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A illustrates an example of a peripheral perfusion assessment system, in accordance with various aspects of the present disclosure.

FIG. 1B illustrates another example of the peripheral perfusion assessment system, in accordance with various aspects of the present disclosure.

FIG. 2 illustrates examples of waveforms generated by blood volume measurement devices, in accordance with various aspects of the present disclosure.

FIG. 3 illustrates a technique for determining features of a blood volume waveform, in accordance with various aspects of the present disclosure.

FIG. 4 illustrates another technique for determining features of a blood volume waveform, in accordance with various aspects of the present disclosure.

FIG. 5 illustrates a systolic time calculation module, in accordance with various aspects of the present disclosure.

FIG. 6 illustrates modules of a peripheral perfusion assessment device, in accordance with various aspects of the present disclosure.

FIG. 7 illustrates an example of a networked-based environment in which some aspects of the present technology may be utilized.

FIG. 8 is a flowchart illustrating a set of operations for assessing peripheral perfusion, in accordance with various aspects of the present disclosure.

FIG. 9 is a diagram showing the flow of data from the measuring device used on the patient ultimately arriving to the monitoring physician.

FIG. 10 is a diagram showing how the monitoring physician can send notifications to the patient through the system according to one embodiment.

FIG. 11 is a diagram showing how the system automatically can send notifications to the patient according to one embodiment.

FIG. 12 is an example of a computer system with which embodiments of the present technology may be utilized.

FIG. 13 is an example of a report generated indicating device output data analysis in interpretable form for the monitoring medical provider. This report allows for interpretation and affords the medical provider to recommend further evaluation and management if necessary. This report is generated in billable form for the provider after interpretation is complete.

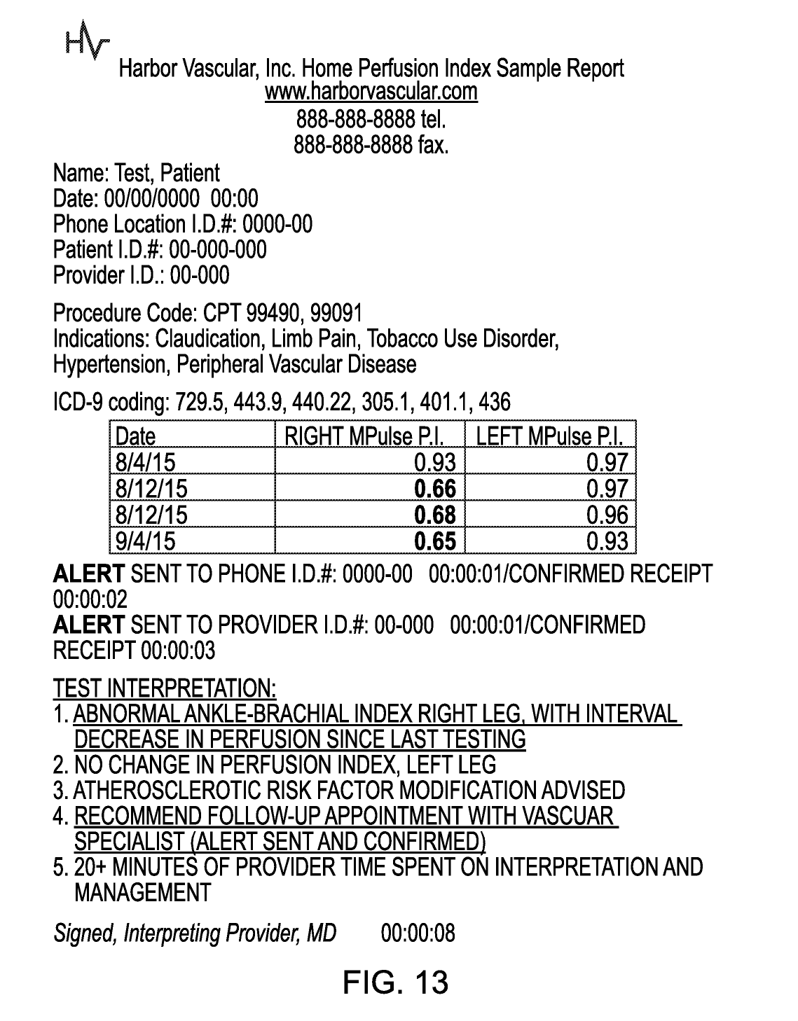

### DETAILED DESCRIPTION

The present technology is directed to apparatuses, systems, and methods for assessing absolute and relative peripheral perfusion. As discussed in greater detail below, two blood volume measurement devices may measure the blood volume at a patient's extremities either simultaneously or in series. Distortions between the waveforms generated by the two blood volume measurement devices are detected, and an assessment of the patient's perfusion is determined based on the degree of distortion. The patient and/or a healthcare provider may be notified if the degree of distortion indicates that the patient is at risk of a peripheral arterial disease.

Some embodiments of the present technology provide for fast and accurate techniques to assess peripheral perfusion in an “at-home” or “out-of-facility” (e.g., telemetric) environment without the use of cuffs. Certain aspects of the present technology provide for continuous measurements of relative peripheral perfusion to detect perfusion deficits and potential limb or life threatening situations.

Blood volume measurement devices, such as plethysmography and photoplethysmography devices, measure changes in the volume of an organ caused by fluctuations in the amount of blood the organ contains. For example, a photoplethysmography device may measure blood volume in the arteries and arterioles of a patient's subcutaneous tissue by illuminating the patient's skin and measuring changes in light absorption. The changes in blood volume may correspond to the cardiac cycle. As the heart pumps blood to the periphery, the arteries and arterioles in the subcutaneous tissue are distended. Thus, blood volume measurement devices can be useful in assessing the peripheral perfusion of a patient. A peripheral perfusion assessment using the blood volume measuring systems and devices disclosed herein can also serve as a surrogate for overall cardiovascular risk assessment.

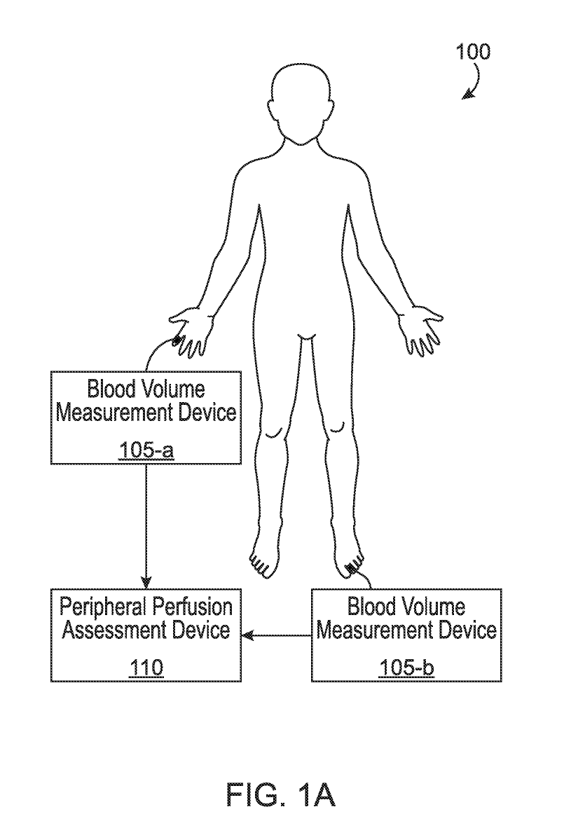

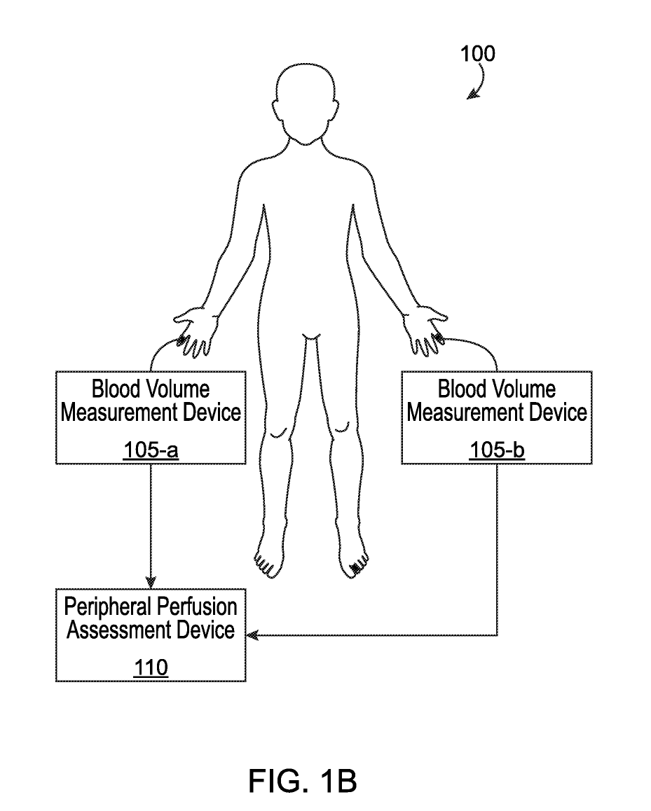

Specific details of several examples of the present technology are described below with reference to FIGS. 1-12. Additionally, several other embodiments of the technology can have different configurations, components, or procedures than those described herein. A person of ordinary skill in the art, therefore, will accordingly understand that the technology can have other embodiments with additional elements and that the technology can have other embodiments without several of the features shown and described below with reference to FIGS. 1-12.

The techniques introduced herein can be embodied as special-purpose hardware (e.g., circuitry), as programmable circuitry appropriately programmed with software and/or firmware, or as a combination of special-purpose and programmable circuitry. Hence, embodiments may include a machine-readable medium having stored thereon instructions that may be used to program a computer (or other electronic devices) to perform a process. The machine-readable medium may include, but is not limited to, floppy diskettes, optical discs, compact disc read-only memories (CD-ROMs), magneto-optical discs, ROMs, random access memories (RAMs), erasable programmable read-only memories (EPROMs), electrically erasable programmable read-only memories (EEPROMs), application-specific integrated circuits (ASICs), magnetic or optical cards, flash memory, or other type of media/machine-readable medium suitable for storing electronic instructions.

### TERMINOLOGY

Brief definitions of terms and phrases used throughout this application are given below.

The terms “connected” or “coupled” and related terms are used in an operational sense and are not necessarily limited to a direct physical connection or coupling. Thus, for example, two devices may be coupled directly, or via one or more intermediary media or devices. As another example, devices may be coupled in such a way that information can be passed there between, while not sharing any physical connection with one another. Based on the disclosure provided herein, one of ordinary skill in the art will appreciate a variety of ways in which connection or coupling exists in accordance with the aforementioned definition.

The phrases “in some embodiments,” “according to some embodiments,” “in the embodiments shown,” “in other embodiments,” and the like generally mean the particular feature, structure, or characteristic following the phrase is included in at least one implementation of the present technology, and may be included in more than one implementation. In addition, such phrases do not necessarily refer to the same embodiments or different embodiments.

If the specification states a component or feature “may”, “can”, “could”, or “might” be included or have a characteristic, that particular component or feature is not required to be included or have the characteristic.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded.

The term “module” or “engine” refers broadly to general or specific-purpose hardware, software, or firmware (or any combination thereof) components. Modules and engines are typically functional components that can generate useful data or other output using specified input(s). A module or engine may or may not be self-contained. Depending upon implementation-specific or other considerations, the modules or engines may be centralized or functionally distributed.

### GENERAL DESCRIPTION

FIG. 1A illustrates an example of a peripheral perfusion assessment system 100, in accordance with various aspects of the present disclosure. The system 100 includes two blood volume measurement devices 105-a and 105-b. The blood volume measurement devices 105-a and 105-b are configured to measure blood volume at two extremities of a patient. For example, as shown in FIG. 1A, blood volume measurement device 105-a is configured to measure blood volume at a finger of the patient, and blood volume measurement device 105-b is configured to measure blood volume at a toe of the patient. The blood volume measurement devices 105-a and 105-b may be plethsymography devices, photoplethysmography devices, or other types of devices for measuring changes in blood volume or blood pressure over time.

The blood volume measurement devices 105-a and 105-b generate continuous waveforms of the blood volume in each extremity. These continuous measurements at each extremity may be taken simultaneously so that the waveforms are aligned in time. The blood volume measurement devices 105-a and 105-b transmit the waveforms of blood volume over time to a peripheral perfusion assessment device 110. In some examples, the peripheral perfusion assessment device 110 may be a component of one or both of the blood volume measurement devices 105-a and 105-b. In other examples, the peripheral perfusion assessment device 110 may be remote from the blood volume measurement devices 105-a and 105-b. The blood volume measurement devices 105-a and 105-b may transmit the waveforms of blood volume over time to the peripheral perfusion assessment device 110 via a wired or wireless connection.

The peripheral perfusion assessment device 110 detects distortions in specific features of the waveforms from the blood volume measurement devices 105-a and 105-b. Based on the distortions, the peripheral perfusion assessment device 110 may identify limb-threatening or life-threatening conditions in peripheral perfusion in the patient. Distortions in peripheral perfusion may be side effects of revascularization with stents, arterial bypass surgery, or other cardiovascular procedures and/or conditions. These distortions can be detected through the continuous analysis techniques described herein, and, in some embodiments, can direct limb-saving medical management for the patient.

FIG. 1B illustrates another example of the peripheral perfusion assessment system 100, in accordance with various aspects of the present disclosure. In this example, blood volume measurement device 105-a is configured to measure blood volume at a first finger of the patient (e.g., a right-hand finger), and blood volume measurement device 105-b is configured to measure blood volume at a second finger of a contralateral upper extremity of the patient (e.g., a left-hand finger). The blood volume measurement devices 105-a and 105-b generate waveforms of the blood volume in each finger, and transmit the waveforms to the peripheral perfusion assessment device 110. The peripheral perfusion assessment device 110 may then detect distortions and identify changes in peripheral perfusion as described in reference to FIG. 1A.

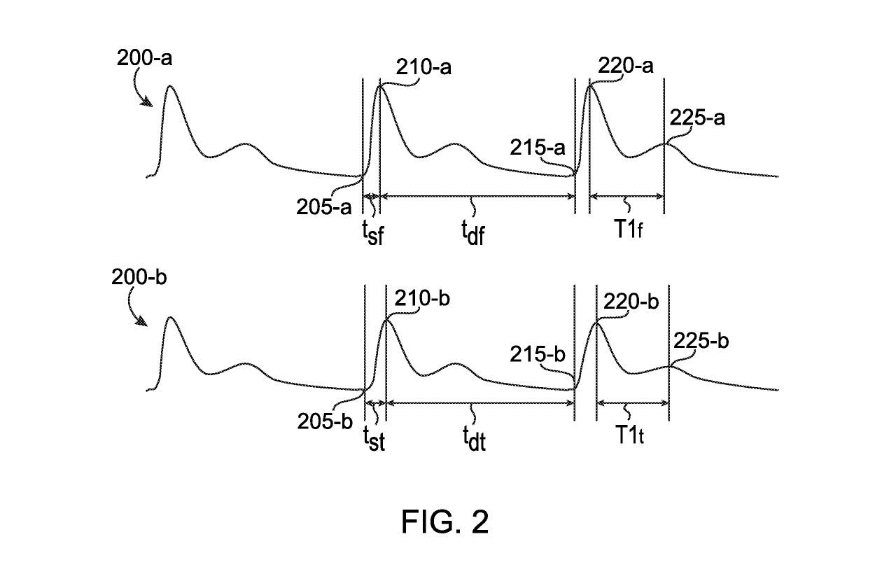

FIG. 2 illustrates examples of waveforms generated by the blood volume measurement devices 105-a and 105-b, in accordance with various aspects of the present disclosure. For example, blood volume measurement device 105-a may generate waveform 200-a, and blood volume measurement device 105-b may generate waveform 200-b. The waveforms 200-a and 200-b may correspond to blood volume measurements taken simultaneously or in close temporal proximity at two different extremity locations of the patient, for example at a patient's finger and toe, respectively. Alternatively, the waveforms 200-a and 200-b may correspond to blood volume measurements taken simultaneously or in close temporal proximity at a patient's right-hand finger and left-hand finger, respectively.

Features of each waveform 200-a and 200-b may correspond to parts of the patient's cardiac cycle. For example, waveform 200-a may include a first diastolic trough 205-a, a first systolic peak 210-a, a second diastolic trough 215-a, a second systolic peak 220-a, and a diastolic peak 225-a, as measured at a patient's finger. A systolic time interval (t_{sf}) may be determined as the time between the first diastolic trough 205-a and the first systolic peak 210-a. A diastolic time interval (t_{df}) may be determined as the time between the first systolic peak 210-a and the second diastolic trough 215-a. An interstolic time interval (T1_{f}) may be determined as the time between second systolic peak 220-a and the diastolic peak 225-a. The waveform 200-b may include similar features, as measured simultaneously or in temporal proximity at a patient's toe.

Relative differences in the systolic time measured at the patient's finger (t_{sf}) and the systolic time measured at a patient's toe (tₛₜ) may indicate a peripheral arterial disease in the patient. Similarly, differences in the systolic time measured at the patient's right-hand finger and the systolic time measured at a patient's left-hand finger may also indicate a peripheral arterial disease in the patient.

In one embodiment, the presence and degree of severity of peripheral arterial disease may be determined by comparing waveform features measured at two extremities. This comparison may be mapped to a digital-digital index (DDI). The value of this DDI relative to a “normal range” may indicate the presence and degree of severity of peripheral arterial disease. In such a case, this may prompt the patient to undergo medical evaluation, confirmatory diagnostic testing and possible therapeutic interventions.

In another embodiment, the degree to which this DDI changes over time may indicate the presence, progression and degree of severity of the disease. This change may be measured in absolute or relative terms. For instance, an absolute drop of about 0.10, about 0.15 or about 0.20 or more, or a relative change of about fifteen percent, about twenty percent, about 25 percent or more over a period of 6 months or more (e.g. about 6 months, about 8 months, about 12 months, or about 24 months) may indicate a severe progression of the disease. It may also be measured in relative terms. Moreover, this relative change over a longer period may indicate significant progression of the disease. In such a case, the patient may need to undergo further testing to determine if any medical therapeutic intervention is required.

In another embodiment, the presence and degree of severity of peripheral arterial disease may be determined by calculating a ratio between the systolic times measured at the two extremities of the patient. For example, a digital-digital index (DDI) ratio may be calculated as

$$DDI = \frac{{p}_{1} {t}_{s 1} + {k}_{1}}{{p}_{2} {t}_{s 2} + {k}_{2}} + {k}_{3} ,$$

where the p coefficients and the k constants are drawn from empirical studies of peripheral arterial diseases (similar to a toe-brachial index—TBI). For measurements taken simultaneously or in temporal proximity at a toe and a finger (“toe-to-finger”), a DDI ratio between 0.65 and 1.0 may indicate low distortion between the two systolic time measurements, and thus a low likelihood of peripheral arterial disease. A toe-to-finger DDI ratio less than 0.35 may suggest, for example, a critical vascular occlusive disease. For measurements taken at a right-hand finger and a left-hand finger (“finger-to-finger”), a DDIf ratio less than 0.9 may suggest, for example, perfusion deficits between the patient's upper extremities, which may signify occlusive disease or a post-surgical arterial steal syndrome. In such a case, the patient may need to undergo further medical evaluation and confirmatory testing to determine if any action should be taken. In one embodiment, if peripheral arterial disease is confirmed, the subject is treated. Treatment can include counseling on lifestyle changes (e.g. smoking, diet, exercise), cholesterol-lowering medications (e.g. statins) to reduce LDL-C to less than 100 mg/dL, blood pressure medication, medication to prevent clot formation (e.g. daily asprin or clopidogrel), or medications to increase blood flow to the limbs such as cilostazol or pentoxifylline. Treatment can also include angioplasty, graft bypass or thrombolytic therapy.

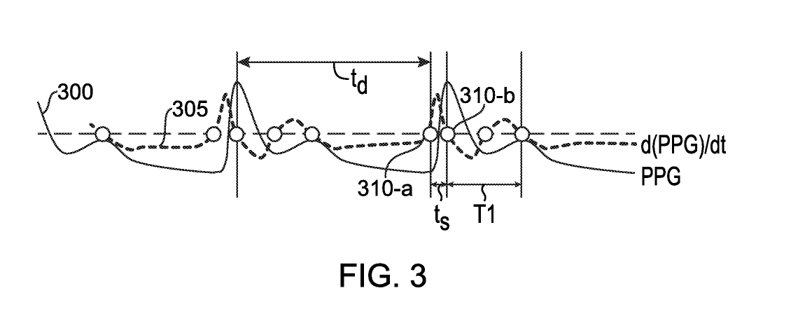

FIG. 3 illustrates an embodiment of a technique for determining features of a blood volume waveform 300, in accordance with various aspects of the present disclosure. A first derivative of the blood volume waveform 300 is calculated, and a derivative waveform 305 is generated. Each of the zero-crossings of the derivative waveform 305 may then be determined. Zero-crossing 310-a corresponds to a diastolic trough of the blood volume waveform 300, and zero-crossing 310-b corresponds to a systolic peak of the blood volume waveform 300. A systolic peak is always higher than a diastolic peak, and a diastolic trough is always lower than a interstolic trough. The systolic time (tₛ) of the blood volume waveform 300 may then be determined as the time interval between zero-crossing 310-a and zero-crossing 310-b.

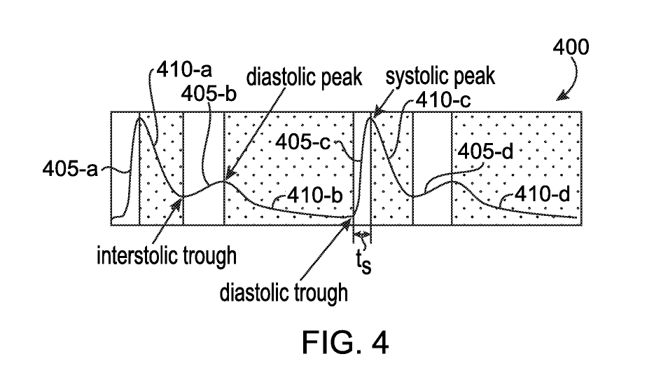

FIG. 4 illustrates another embodiment of a technique for determining features of a blood volume waveform 400, in accordance with various aspects of the present disclosure. The blood volume waveform is divided into increasing segments 405 (light background), and decreasing segments 410 (gray backgrounds). The increasing segments 405 correspond to segments where the first derivative of the blood volume waveform 400 is positive. The decreasing segments 410 correspond to segments where the first derivative of the blood volume waveform 400 is negative. The peaks of the blood volume waveform 400 occur where the first derivative changes from positive to negative. For example, a systolic peak occurs between increasing segment 405-a and decreasing segment 410-a, and a diastolic peak occurs between increasing segment 405-b and decreasing segment 410-b. The troughs of the blood volume waveform 400 occur where the first derivative changes from negative to positive. For example, an interstolic trough occurs between decreasing segment 410-a and increasing segment 405-b, and a diastolic trough occurs between decreasing segment 410-b and increasing segment 405-c. Thus, the primary features of a blood volume waveform 400 may be determined using the sign changes of a first derivative of the blood volume waveform 400. The actual values of the first derivative may be ignored. The systolic time of the blood volume waveform 400 may then be determined as the time interval between the diastolic trough and the systolic peak.

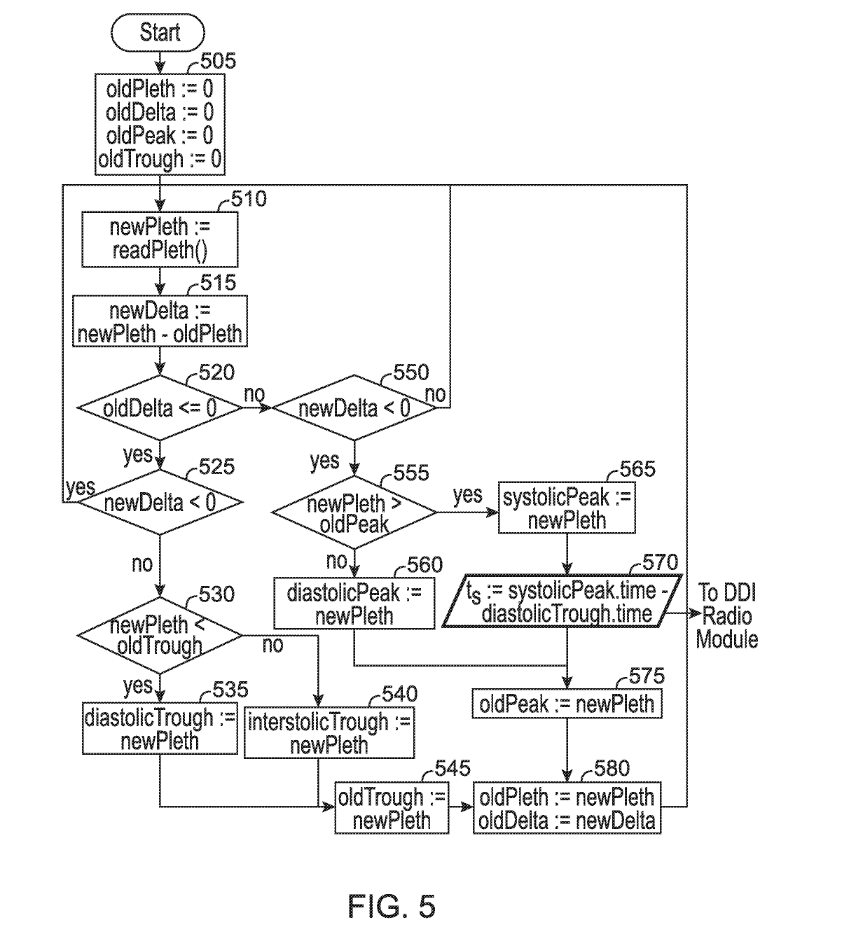

FIG. 5 illustrates an embodiment of a systolic time calculation module 500, in accordance with various aspects of the present disclosure. The systolic time calculation module 500 determines the systolic time from an input blood volume waveform (also referred to as a plethysmograph). The systolic time calculation module 500 may be anywhere in the system including but not limited to a component of the peripheral perfusion assessment device 110, the mobile device, internet service, internet application or computer or device used by the physician. The systolic time calculation module 500 calculates the systolic time of the input blood volume waveform by determining where the waveform changes from an increasing segment to a decreasing segment, or changes from a decreasing segment to an increasing segment, as described in reference to FIG. 4. If the value of the blood volume measurement where the waveform changes from an increasing segment to a decreasing segment is larger than other increasing-to-decreasing values, then the systolic time calculation module 500 determines that value to be a systolic peak, and notes the time when the systolic peak occurred. If the value of the blood volume measurement where the waveform changes from a decreasing segment to an increasing segment is lower than other decreasing-to-increasing values, then the systolic time calculation module 500 determines that value to be a diastolic trough, and notes the time when the diastolic trough occurred. The systolic time calculation module 500 may then determine the time interval between the systolic peak and the previous diastolic trough, and output the time interval as the systolic time of the input blood volume waveform.

The flowchart of FIG. 5 further illustrates a method by which the systolic time calculation module 500 determines the systolic time of an input blood volume waveform in accordance with an embodiment of the present disclosure. At step 505, the values oldPleth, oldDelta, oldPeak, and oldTrough used by the module 500 are initialized to zero. At step 510, the module 500 reads a blood volume value from the input waveform, and stores it as newPleth. The module 500 then calculates the difference between newPleth and oldPleth, and stores the result as newDelta at step 515. At step 520, the module 500 determines if the value oldDelta is less than or equal to zero. If yes, then the module 500 determines if newDelta is less than zero at step 525. If yes, the module 500 returns to step 510 to read another blood volume value and store it as newPleth. If newDelta is greater than or equal to zero at step 525, then the module 500 determines if newPleth is also less than oldTrough at step 530. If not, then the module 500 stores the value of newPleth as interstolicTrough at step 540, and also stores the value of newPleth as oldTrough at step 545. At step 580, the module further stores the value of newPleth as oldPleth and the value of newDelta as oldDelta. The module 500 then returns to step 510 to read another blood volume value and store it as newPleth. Returning to step 530, if newPleth is less than oldTrough, then the module 500 proceeds to step 535 and stores the value of newPleth as diastolicTrough. The module 500 then proceeds to step 545 and continues as described above.

Returning to step 520, if oldDelta is greater than zero, then the module 500 proceeds to step 550 and determines if newDelta is less than zero. If not, then the module 500 returns to step 510 to read another blood volume value and store it as newPleth. If newDelta is less than zero in step 550, then module 500 determines if newPleth is greater than oldPeak in step 555. If yes, then the module 500 stores the value of newPleth as systolic Peak at step 565. The module 500 then determines the time corresponding to the diastolicTrough value and the time corresponding to the systolicPeak value, and calculates the difference between the two times. The module 500 then outputs the difference as the systolic time (tₛ) of the input blood volume waveform. The module 500 then stores the value of newPleth as oldPeak at step 575. At step 580, the module 500 further stores the value of newPleth as oldPleth and the value of newDelta as oldDelta. The module 500 then returns to step 510 to read another blood volume value and store it as newPleth.

Returning to step 555, if newPleth is less than or equal to oldPeak, then module 500 proceeds to step 560 and stores the value of newPleth as distolicPeak. At step 575, the module also stores the value of newPleth as oldPeak, and then further stores the value of newPleth as oldPleth and the value of newDelta as oldDelta at step 580. The module 500 then returns to step 510 to read another blood volume value and store it as newPleth. The module 500 continues reading in new blood volume values until a predetermined number of blood volume values have been processed, a predetermined time interval of the blood volume waveform has been processed, or all values of the blood volume waveform have been processed.

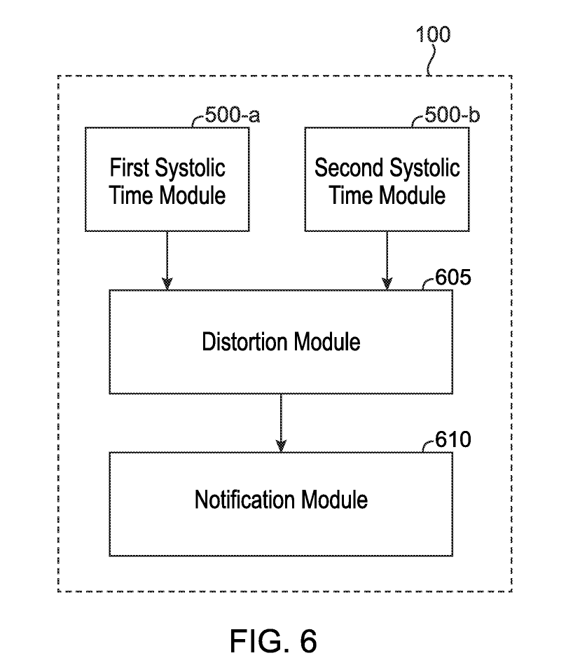

FIG. 6 illustrates modules of a peripheral perfusion assessment device 110, in accordance with various aspects of the present disclosure. A first systolic time module 500-a may receive a first blood volume waveform from a first blood volume measurement device 105-a. A second systolic time module 500-b may receive a second blood volume waveform from a second blood volume measurement device 105-b. The first and second blood volume waveforms may correspond to blood volumes measured simultaneously at two extremities of a patient. The first systolic time module 500-a determines a systolic time of the first blood volume waveform, and the second systolic time module 500-b determines a systolic time of the second blood volume waveform. The first and second systolic time modules 500-a and 500-b may determine the systolic times using the method illustrated in FIG. 5, or using other systolic time calculation methods. In some examples, the peripheral perfusion assessment device 110 may include a single systolic module 500, and may calculate both the systolic time of the first blood volume waveform and systolic time of the second blood volume waveform.

A distortion module 605 receives the two systolic times from the systolic time modules 500-a and 500-b, and determines a degree of distortion between the first blood volume waveform and the second blood volume waveform. The degree of distortion may be determined using the DDI ratio described in reference to FIG. 2, or may be determined using other comparison methods. Based on the degree of distortion between the first and second blood volume waveforms, the distortion module 605 assesses the patient's peripheral perfusion. Based on the assessment of the distortion module 605, a notification module 610 may notify the patient, a healthcare provider, or both of the assessment. If the distortion module 605 assesses that the patient is at risk of a peripheral arterial disease, then the notification may instruct the patient and/or healthcare provider of the need for medical attention. If the distortion module 605 assesses that the patient is not at risk of a peripheral arterial disease, then the notification may instruct the patient and/or healthcare provider of the current status of the patient's peripheral perfusion. The notification module 610 may also store and/or transmit the blood volume waveforms, the systolic times, the DDI ratio, and/or other measurements for further processing and/or analysis. The notification module 610 may notify the patient of the assessment via a display, an alarm, a haptic interface, or other types of notifications. The notification module 610 may notify the healthcare provider via a wired or wireless connection to a healthcare provider device.

In some examples, the notification module 610 may directly receive the blood volume waveforms from the first and second blood volume measurement devices 105-a and 105-b. The notification module 610 may then transmit the blood volume waveforms to another device for analysis and assessment of the patient's peripheral perfusion. For example, the notification module 610 may transmit the blood volume waveforms to a healthcare provider device.

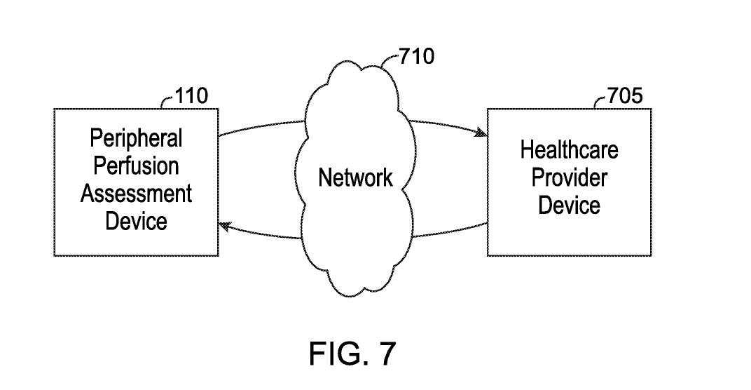

FIG. 7 illustrates an example of a networked-based environment in which some aspects of the present technology may be utilized. As shown in FIG. 7, a peripheral perfusion assessment device 110 communicates with a healthcare provider device 705 via a network 710. The peripheral perfusion assessment device 110 and healthcare provider device 705 may be conventional computer systems (e.g., a desktop or laptop computer), mobile devices having computer functionality (e.g., a mobile telephone, a smartphone, wearable computer, etc.), or purpose-built computing devices for communicating peripheral perfusion assessments.

The peripheral perfusion assessment device 110 and healthcare provider device 705 can be configured to use network 710 to communicate. In accordance with various embodiments, network 710 can include any combination of local area and/or wide area networks, using both wired and wireless communication systems. In one embodiment, network 710 uses standard communications technologies and/or protocols. Thus, network 710 may include links using technologies such as Ethernet, 802.11, Bluetooth, near-field communications (NFC), worldwide interoperability for microwave access (WiMAX), 3G, 4G, CDMA, digital subscriber line (DSL), etc. Similarly, the networking protocols used on network 710 may include multiprotocol label switching (MPLS), transmission control protocol/Internet protocol (TCP/IP), User Datagram Protocol (UDP), hypertext transport protocol (HTTP), simple mail transfer protocol (SMTP) and file transfer protocol (FTP). Data exchanged over network 710 may be represented using technologies and/or formats including hypertext markup language (HTML) or extensible markup language (XML). In addition, all or some links can be encrypted using conventional encryption technologies such as secure sockets layer (SSL), transport layer security (TLS), and Internet Protocol security (IPsec).

In some embodiments, the peripheral perfusion assessment device 110 and healthcare provider device 705 can retrieve or submit information to one another. For example, the peripheral perfusion assessment device 110 may transmit a notification of a peripheral perfusion assessment, a DDI ratio, blood volume waveforms, and/or other measurements from the blood volume measurement devices 105-a and 105-b to the healthcare provider device 705. The healthcare provider device 705 may transmit a notification of a peripheral perfusion assessment, warning, and/or other instructions from the healthcare provider to the peripheral perfusion assessment device 110. In these examples, the peripheral perfusion assessment device 110 may notify the patient of the assessment, warnings, and/or instructions from the healthcare provider device 705. In some examples, the healthcare provider device 705 may store historical records of the peripheral perfusion assessments, DDI ratios, blood volume waveforms, and/or other measurements from one or more patients in a database. The healthcare provider device 705 may use the historical records database for further analysis of one or more patients' health data. The database may include various database components that can be implemented in the form of a database that is relational, sequential, hierarchical, scalable, secure, and/or featuring other attributes. Examples of such database include, but are not limited to, DB2, MySQL, Oracle, Sybase, and the like. Alternatively, these databases may be implemented using various standard data-structures, such as an array, hash, list, struct, structured text file (e.g., XML), table, binary, and/or the like. Such data structures may be stored in memory and/or in structured files.

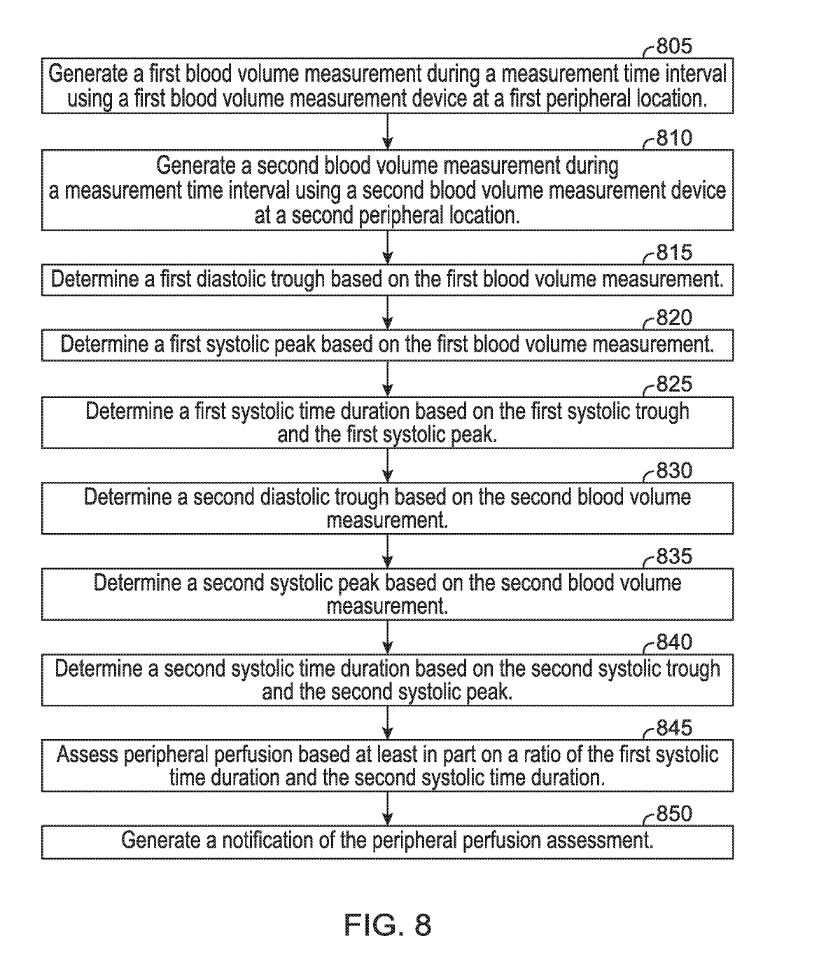

FIG. 8 is a flowchart illustrating a set of operations for assessing peripheral perfusion, in accordance with various aspects of the present disclosure. The operations illustrated in FIG. 8 may be executed by a blood volume measurement device 105, a peripheral perfusion assessment device 110, a healthcare provider device 705, and/or a combination of devices. The devices may include a memory and one or more processors. These components are examples of various means for performing some of the operations illustrated in FIG. 8.

At block 805, the set of operations include generating a first blood volume measurement during a measurement time interval using a first blood volume measurement device at a first peripheral location. For example, the first peripheral location may be a toe of a patient. At block 810, the set of operations include generating a second blood volume measurement during a measurement time interval using a second blood volume measurement device at a second peripheral location. For example, the second peripheral location may be a finger of the patient. The first blood volume measurement device and the second blood volume measurement device may include plethysmography or photoplethysmography devices.

At block 815, the set of operations include determining a first diastolic trough based on the first blood volume measurement. At block 820, the set of operations include determining a first systolic peak based on the first blood volume measurement. The first systolic peak and the first diastolic trough may be determined based on a first derivative of the first blood volume measurement. At block 825, the set of operations include determining a first systolic time duration based on the first diastolic trough and the first systolic peak. At block 830, the set of operations include determining a second diastolic trough based on the second blood volume measurement. At block 835, the set of operations include determining a second systolic peak based on the second blood volume measurement. The second systolic peak and the second diastolic trough may be determined based on a first derivative of the second blood volume measurement. At block 840, the set of operations include determining a second systolic time duration based on the second diastolic trough and the second systolic peak. At block 845, the set of operations include assessing peripheral perfusion based at least in part on a ratio of linear functions of the first systolic time duration and the second systolic time duration. At block 850, the set of operations include generating a notification of the peripheral perfusion assessment. The notification may then be transmitted to a healthcare provider.

### Flow of Telemetry Data to Physician Overview

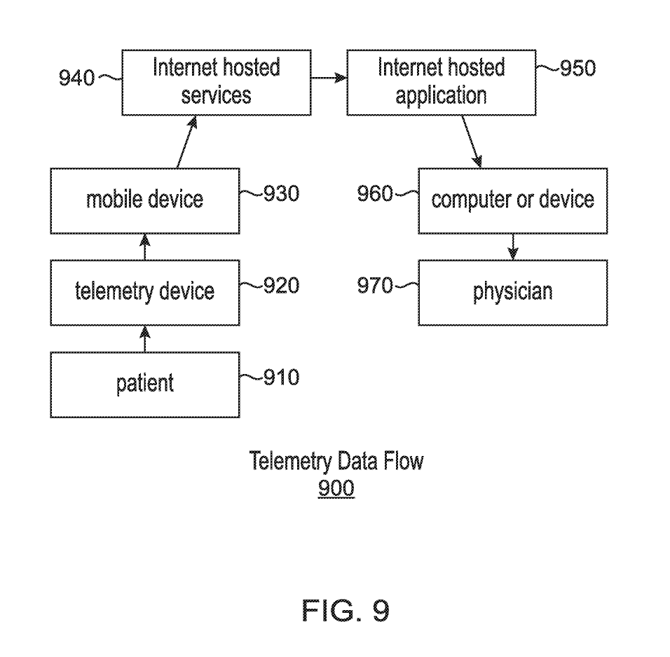

Embodiments of the present technology include paths along which the patient's measurements and other data travel on the way to a monitoring physician. These paths include various way points where the data may be stored, transformed, formatted or aggregated with other data and/or transmitted. FIG. 9 is an example of such a path. The number of waypoints and the actions taken at each way point may vary from that described in FIG. 9. According to the present example, one or more telemetry devices 920 is applied to the patient 910. The telemetry device captures patient data and sends them to a mobile device 930. The mobile may store these data, perform calculations on them, format them, aggregate them with other data, etc. and send data to Internet hosted services 940, which also may store the data, transform them, aggregate them, etc. Internet hosted applications 950 may serve these resulting data to a computer or device 960 used by a monitoring physician 970.

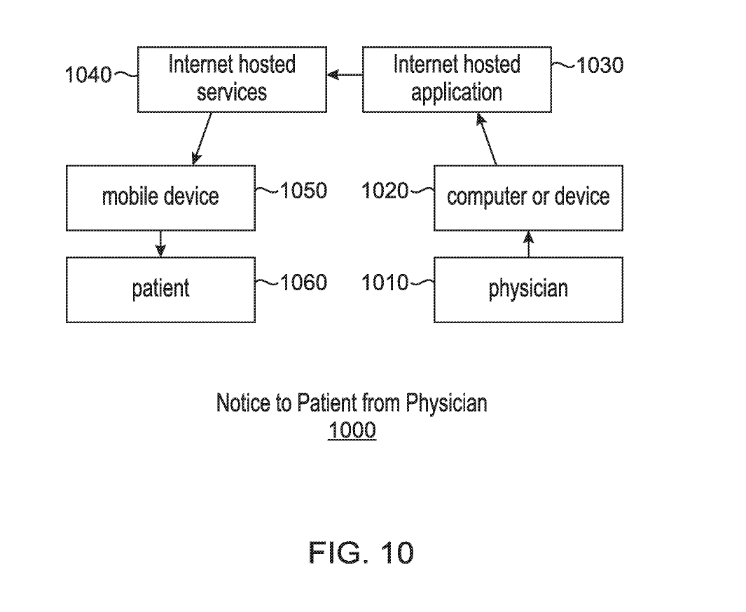

Embodiments of the present technology include paths along which the physician may notify the patient for reasons including but not limited to a reminder to take a new reading. These paths include various way points where the notification may be stored, transformed, formatted or aggregated with other data and/or transmitted. FIG. 10 is an example of such a path. The number of waypoints and the actions taken at each way point may vary from that described in FIG. 10. According to the present example, a monitoring physician 1010 uses a computer or device 1020 to initiate a notification to the patient. This computer or device sends the request to an internet hosted application 1030 which sends a request to an internet hosted service 1040 which, in turn, delivers a notification to a mobile device 1050 which reaches the patient 1060.

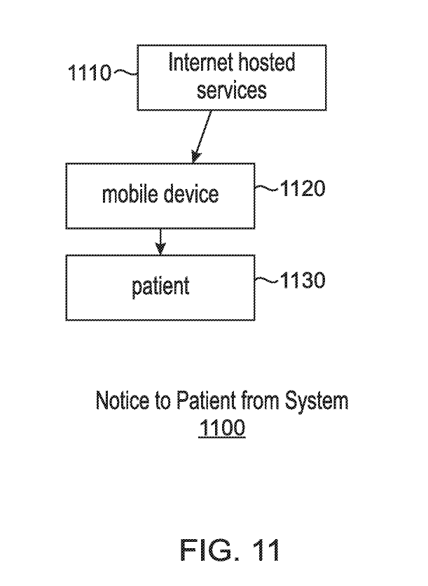

Embodiments of the present technology include paths along which the system may automatically notify the patient for reasons including but not limited to a reminder to take a new reading. These paths include various way points where the notification may be stored, transformed, formatted or aggregated with other data and/or transmitted. FIG. 11 is an example of such a path. The number of waypoints and the actions taken at each way point may vary from that described in FIG. 11. According to the present example, an internet hosted application 1110 decides to initiate a notification to the patient based on some criteria possibly including but not limited to the last known telemetry reading from that patient. This internet hosted service sends a notification to a mobile device 1120 which reaches the patient 1130.

### Computer System Overview

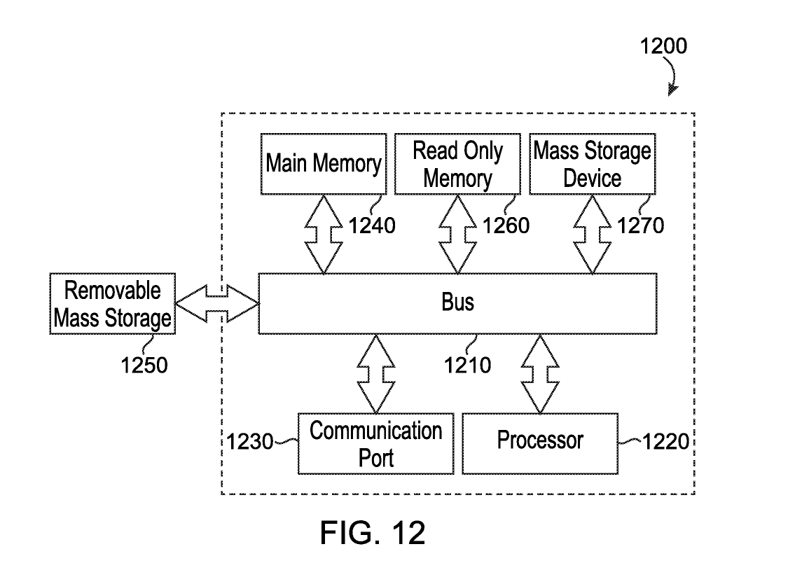

Embodiments of the present technology include various steps and operations, which have been described above. A variety of these steps and operations may be performed by hardware components or may be embodied in machine-executable instructions, which may be used to cause a general-purpose or special-purpose processor programmed with the instructions to perform the steps. Alternatively, the steps may be performed by a combination of hardware, software, and/or firmware. As such, FIG. 12 is an example of a computer system 1200 with which embodiments of the present technology may be utilized. According to the present example, the computer system includes a bus 1210, at least one processor 1220, at least one communication port 1230, main memory 1240, a removable storage media 1250, a read only memory 1260, and a mass storage 1270.

Processor(s) 1220 can be any known processor, such as, but not limited to, Intel® lines of processor(s); AMD® lines of processor(s); ARM® lines of processors, or other application-specific integrated circuits (ASICs). Communication port(s) 1230 can be any communication port, such as, but not limited to, an RS-232 port for use with a modem-based dialup connection, a 10/100 Ethernet port, a Gigabit port using copper or fiber, wireless antennas, etc. Communication port(s) 1230 may be chosen depending on a network such as a Local Area Network (LAN), Wide Area Network (WAN), cellular network, or any network to which the computer system 1200 connects.

Main memory 1240 can be Random Access Memory (RAM) or any other dynamic storage device(s) commonly known in the art. Read only memory 1260 can be any static storage device(s) such as Programmable Read Only Memory (PROM) chips for storing static information such as instructions for processor 1220.

Mass storage 1270 can be used to store information and instructions. For example, hard disks such as the Adaptec® family of SCSI drives, an optical disc, an array of disks such as RAID or such as the Adaptec family of RAID drives, or any other mass storage devices may be used.

Bus 1210 communicatively couples processor(s) 1220 with the other memory, storage and communication blocks. Bus 1210 can be any system communication bus, such as, but limited to, I2C, PCI, PCI-Express, UMI, DMI, QPI, etc.

Removable storage media 50 can be any kind removable storage, such as, but not limited to, external hard-drives, flash memory cards, floppy drives, Compact Disc-Read Only Memory (CD-ROM), Compact Disc-Re-Writable (CD-RW), Digital Video Disk-Read Only Memory (DVD-ROM), Blu-Ray, etc.

The components described above are meant to exemplify some types of possibilities. In no way should the aforementioned examples limit the scope of the technology, as they are only embodiments.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments. All references cited herein are incorporated by reference as if fully set forth herein.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

We claim:

1. A method for assessing peripheral perfusion in a subject, comprising:
generating a first blood volume measurement during a measurement time interval using a first blood volume measurement device at a first peripheral location of the subject;
generating a second blood volume measurement during a measurement time interval using a second blood volume measurement device at a second peripheral location of the subject;
determining a first diastolic trough based on the first blood volume measurement;
determining a first systolic peak based on the first blood volume measurement;
determining a first systolic time duration based on the first diastolic diastolic trough and the first systolic peak;
determining a second diastolic trough based on the second blood volume measurement;
determining a second systolic peak based on the second blood volume measurement;
determining a second systolic time duration based on the second diastolic trough and the second systolic peak; and
assessing peripheral perfusion based at least in part on a ratio of linear functions of the first systolic time duration and the second systolic time duration.

2. The method of claim 1, wherein:
determining the first diastolic trough and the first systolic peak comprises determining a first derivative of the first blood volume measurement, and
determining the second diastolic trough and the second systolic peak comprises determining a first derivative of the second blood volume measurement.

3. The method of claim 2, wherein the first diastolic trough and the first systolic peak are determined based on a sign of the first derivative of the first blood volume measurement, and the second diastolic trough and the second systolic peak are determined based on a sign of the first derivative of the second blood volume measurement.

4. The method of claim 1, wherein the first blood volume measurement device and the second blood volume measurement device comprise plethysmography devices.

5. The method of claim 1, wherein the first blood volume measurement device and the second blood volume measurement device comprise photoplethysmography devices.

6. The method of claim 1, wherein the first peripheral location comprises a toe of a patient, and the second peripheral location comprises at least one of a finger of the patient or a finger of the contralateral upper extremity of the patient.

7. The method of claim 1, assessing peripheral perfusion comprises:
determining the ratio of the first systolic time duration and the second systolic time duration indicates one or more of a peripheral arterial disease and a perfusion deficit.

8. The method of claim 7 further comprising recommending for treatment for or treating the subject for peripheral arterial disease or a perfusion deficit or conducting further tests to confirm peripheral arterial disease or a perfusion deficit.

9. The method of claim 8 wherein the treatment is selected from cholesterol-lowering medication, blood pressure medication, medication to prevent clot formation, medication to increase blood flow to the limbs, angioplasty, graft bypass or thrombolytic therapy.

10. The method of claim 1, further comprising:
generating a notification of the peripheral perfusion assessment.

11. The method of claim 10, further comprising:
displaying the notification.

12. The method of claim 11, further comprising:
transmitting the notification to a healthcare provider.

13. A system for assessing peripheral perfusion, comprising:
a first blood volume measurement device for generating a first blood volume measurement during a measurement time interval at a first peripheral location;
a second blood volume measurement device for generating a second blood volume measurement during a measurement time interval at a second peripheral location;
a peripheral perfusion assessment device for receiving the first blood volume measurement and the second blood volume measurement, wherein the peripheral perfusion assessment device:
determines a first diastolic trough based on the first blood volume measurement;
determines a first systolic peak based on the first blood volume measurement;
determines a first systolic time duration based on the first diastolic trough and the first systolic peak;
determines a second diastolic trough based on the second blood volume measurement;
determines a second systolic peak based on the second blood volume measurement;
determines a second systolic time duration based on the second diastolic trough and the second systolic peak; and
assesses peripheral perfusion based at least in part on a ratio of linear functions of the first systolic time duration and the second systolic time duration.

14. The system of claim 13 further configured to deliver patient-related telemetry data to a physician.

15. The system of claim 14 further configured to deliver notifications to the patient when initiated by the physician.

16. The system of claim 15 wherein the notifications are automatically initiated.
